(19) United States
(12) Patent Application Publication
(10) Pub. No.: US 2010/0167984 A1
(43) Pub. Date: Jul. 1, 2010

(54) Complex between human insulin and an amphiphilic polymer and use of this complex in the preparation of a fast-acting human insulin formulation

(75) Inventors: Olivier Soula, Meyzieu (FR); Remi Soula, Lyon (FR); Gerard Soula, Meyzieu (FR)

Correspondence Address: OLIFF & BERRIDGE, PLC, P.O. BOX 320850, ALEXANDRIA, VA, 22320-4850, US

(73) Assignee: ADOCIA, LYON (FR)

(21) Appl. No.: 12/588,349

(22) Filed: Oct. 13, 2009

Related U.S. Application Data
(60) Provisional application No. 61/136,894, filed on Oct. 10, 2008.

(30) Foreign Application Priority Data
Sep. 26, 2008 (FR) 08/05321
Oct. 10, 2008 (FR) 08 56896

(51) Int. Cl. A61K 38/28; C07K 14/62

(52) U.S. Cl. 514/3; 530/303

(57) ABSTRACT

The invention relates to a complex between human insulin and an amphiphilic polymer comprising carboxyl functional groups, said amphiphilic polymer being chosen from functionalized polysaccharides predominantly composed of glycoside monomers bonded via glycoside bonds of (1,6) type which are functionalized by at least one tryptophan derivative.

It also relates to a pharmaceutical composition comprising at least one complex according to the invention, it being possible for said formulation to be in the form of an injectable solution.

It more particularly relates to the use of a complex according to the invention in the preparation of a human insulin formulation at a concentration of approximately 600 μM (100 IU/ml), the onset of action of which is less than 30 minutes, preferably less than 20 minutes and more preferably less than 15 minutes and/or the glycemic nadir of which is at less than 120 minutes, preferably less than 105 minutes and more preferably less than 90 minutes.

The present invention relates to a stable fast-action recombinant human insulin formulation.

Since the production of insulin by genetic engineering at the beginning of the 1980s, diabetic patients have benefited from human insulin in their treatment. This product has greatly improved this therapy since the immunological risks related to the use of non-human insulin, in particular porcine insulin, are found to be eliminated.

Genetic engineering has made possible another improvement in the treatment of diabetes with the development of insulin “analogs”. These insulins are modified in order to achieve two complementary objectives:
on the one hand, to have a slow and controlled action over 24 hours: this is the case with Lantus, which has a much better prolonged action than that of human insulin;
on the other hand, a very fast action after injection: this is the case with the insulins Lispro (Lilly), Novolog (Novo) and Apidra (Aventis), which have rates of action after administration which are superior to that of human insulin.

The control of the rate of action of insulin is a crucial element in the life of patients as they have, at each meal, to avoid situations where they might end up in a hyperglycemic state or hypoglycemic state. It is therefore of the highest importance medically to cause to coincide, as much as can be done, the action of the insulin administered with the production of glucose contributed by the foodstuffs. It is this which justifies today the success of ultrarapid insulin analogs at the expense of the use of human insulin.

These insulins are modified on one or two amino acids in order to be more rapidly active after a subcutaneous injection. These insulins, Lispro (Lilly), Novolog (Novo) and Apidra (Aventis), are stable solutions of insulin with a hypoglycemic response similar in terms of kinetics to the physiological response generated by the beginning of a meal. Consequently, the patients no longer have to plan their mealtime before the injection of a fast-acting insulin. They inject themselves with this insulin at a time when they are ready to eat. They can even, if necessary, supplement this dose at the end of their meal, which is very nice for children, for whom it is difficult to adjust the dose and to control the appetite.

Human insulin does not make it possible to obtain a hypoglycemic response similar in terms of kinetics to the physiological response generated by the beginning of a meal as it is assembled in the hexamer form whereas it is active in the monomer and dimer forms. The equilibria for dissociation of the hexamers to give dimers and of the dimers to give monomers slow down its action by approximately 20 minutes in comparison with a fast-acting insulin analog, Brange J. et al., Advanced Drug Delivery Review, 35,1999, 307-335. Human insulin is prepared in the form of hexamers in order to be stable for approximately 2 years at 4° C. as, in the form of monomers, it has a very strong propensity to aggregate and then to fibrillate, which causes it to lose its activity; furthermore, in this aggregated form, it exhibits an immunological risk to the patient.

The principle of “fast-acting” insulin analogs is to form hexamers, in order to ensure the stability of the insulin, but also to promote the dissociation of the hexamers to give monomers in order to obtain a fast action.

The main disadvantage of these insulins is the modification to the primary structure of human insulin. This modification brings about variations in interaction with the insulin receptors present on a very large number of cell lines as it is known that the role of insulin in the body is not limited solely to its hypoglycemic activity. Although many research studies have been carried out in this field, it is to date not known how to determine if these insulin analogs have all the physiological properties of human insulin.

Furthermore, the number of diabetic patients is increasing daily. It is of the highest importance to provide patients affected by this disease with insulin formulations which are as cheap as possible. There thus exists a real and unsatisfied need for a fast-acting human insulin formulation which is more effective, safer and cheaper than the current formulations on the market, which are either fast-acting insulin analogs or human insulins which have an excessively long action time.

Biodel has provided a solution to this problem by adding EDTA and citric acid to human insulin. Their strategy is thus to destabilize the hexamer by taking up an acidic pH and by complexing the zinc ions by the EDTA. However, such a formulation exhibits several major disadvantages. The first, due to the acidity of the formulation, is that the human insulin solution has to be prepared at the time of the injection, this being the case several times daily, whereas today patients are using stable ready-for-use solutions which can be administered by insulin pens. The second is that, in order to obtain the desired effects, the solution tested by Biodel is four time more dilute than the international standard, which is 100 IU/ml for all the insulin solutions on the market. The third is a high frequency of pain at the injection site, which pain is attributed to the large volume of liquid injected, as revealed in the phase III clinical studies.

The present invention makes it possible to solve the various problems set out above since it makes it possible to prepare a human insulin formulation which is stable at a pH of between 5.5 and 7.5 in solution at 100 IU/ml, said formulation making it possible to achieve, after administration, a plasma level of insulin and/or a reduction in the glucose more rapidly than with human insulin formulations.

The invention consists in forming a complex of human insulin with an amphiphilic polymer comprising carboxyl functional groups.

This complex can furthermore be formed by simply mixing an aqueous solution of insulin and an aqueous solution of amphiphilic polymer.

The invention also relates to the complex between human insulin and an amphiphilic polymer comprising carboxyl functional groups.

It also relates to the use of this complex in preparing human insulin formulations which make it possible to achieve, after administration, a plasma level of insulin and/or a reduction in the glucose more rapidly than human insulin formulations.

The “fast-acting” human insulin formulations on the market at a concentration of 600 μM (100 IU/ml) have an onset of action of between 30 and 60 minutes and a glycemic nadir at between 2 and 4 hours.

The “fast-acting” insulin analog formulations on the market at a concentration of 600 μM (100 IU/ml) have an onset of action of between 10 and 15 minutes and a glycemic nadir at between 60 and 90 minutes.

The invention relates more particularly to the use of a complex according to the invention in the preparation of a “fast-acting” insulin formulation.

The invention relates to the use of the complex according to the invention in preparing human insulin formulations at a concentration of approximately 600 μM (100 IU/ml), the onset of action of which is less than 30 minutes, preferably less than 20 minutes and more preferably still less than 15 minutes.

The invention relates to the use of the complex according to the invention in preparing human insulin formulations at a concentration of approximately 600 μM (100 IU/ml), the glycemic nadir of which is at less than 120 minutes, preferably less than 105 minutes and more preferably less than 90 minutes.

In one embodiment, the amphiphilic polymer comprising carboxyl functional groups is chosen from functionalized polysaccharides predominantly composed of glycoside monomers bonded via glycoside bonds of (1,6) type and, in one embodiment, the polysaccharide predominantly composed of glycoside monomers bonded via glycoside bonds of (1,6) type is a functionalized dextran comprising carboxyl functional groups.

Said polysaccharides are functionalized by at least one tryptophan derivative, denoted Trp:
said tryptophan derivative being grafted or bonded to the polysaccharides by coupling with an acid function, said acid function being an acid function carried by a connecting arm R bonded to the polysaccharide via a function F, said function F resulting from the coupling between the connecting arm R and an —OH function of the polysaccharide,
F being either an ester, thionoester, amide, carbonate, carbamate, ether, thioether or amine function,
R being a chain comprising between 1 and 6 carbons, optionally branched and/or unsaturated, comprising one or more heteroatoms, such as O, N and/or S, and having at least one carboxyl functional group,
Trp being a residue of an L or D tryptophan derivative, the product of the coupling between the amine of the tryptophan and at least one acid carried by the R group and/or one acid carried by the polysaccharide comprising carboxyl functional groups.

According to the invention, the functionalized polysaccharides can correspond to the following general formula:

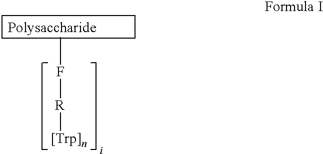

F resulting from the coupling between the connecting arm R and an —OH function of the polysaccharide and being either an ester, thionoester, amide, carbonate, carbamate, ether, thioether or amine function,
R being a chain comprising between 1 and 6 carbons, optionally branched and/or unsaturated, comprising one or more heteroatoms, such as O, N and/or S, and having at least one carboxyl function,
Trp being a residue of an L or D tryptophan derivative, the product of the coupling between the amine of the tryptophan derivative and at least one acid carried by the R group and/or one acid carried by the polysaccharide comprising carboxyl functional groups,
   n represents the molar fraction of the R groups substituted by Trp and is between 0.05 and 0.7, preferably 0.1 and 0.5, more preferably 0.3 and 0.4,
   i represents the molar fraction of the F-R-[Trp]n groups carried per saccharide unit and is between 0 and 2,
      when R is not substituted by Trp, then the acid or acids of the R group are carboxylates of a cation, preferably an alkali metal cation, such as Na+ or K+,
      said polysaccharides being amphiphilic at neutral pH.

In one embodiment, the polysaccharide is a dextran.

In one embodiment, F is either an ester, a carbonate, a carbamate or an ether.

In one embodiment, the polysaccharide according to the invention is characterized in that the group R is chosen from the following groups:

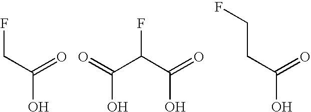

or their salts of alkali metal cations.

In one embodiment, the polysaccharide according to the invention is characterized in that the tryptophan derivative is chosen from the group consisting of tryptophan, tryptophanol, tryptophanamide, 2-indoleethylamine and their alkali metal cation salts.

In one embodiment, the polysaccharide according to the invention is characterized in that the tryptophan derivative is chosen from tryptophan esters of formula II:

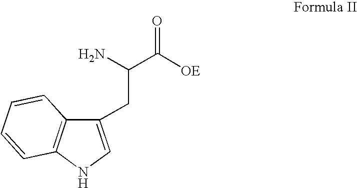

E being a linear or branched C1 to C4 alkyl group.

The polysaccharide can have a degree of polymerization of between 10 and 3000.

In one embodiment, it has a degree of polymerization of between 10 and 400.

In another embodiment, it has a degree of polymerization of between 10 and 200.

In another embodiment, it has a degree of polymerization of between 10 and 50.

In one embodiment, the insulin is a recombinant human insulin as described in the European Pharmacopoeia.

In one embodiment, the polymer/insulin ratios by weight are between 0.1 and 5.

In one embodiment, they are between 0.5 and 2.2.

In one embodiment, they are between 0.7 and 1.3.

Preferably, this composition is in the form of an injectable solution.

In one embodiment, the concentration of insulin in the solutions is 600 μM, i.e. 100 IU/ml.

In one embodiment, the concentration of insulin of 600 μM can be reduced by simple dilution, in particular for pediatric applications.

The invention also relates to a pharmaceutical composition according to the invention, characterized in that it is obtained by drying and/or lyophilization.

In the case of local and systemic releases, the methods of administration envisaged are intravenously, subcutaneously, intradermally, transdermally, intramuscularly, orally, nasally, vaginally, ocularly, buccally, pulmonary, and the like.

The invention also relates to the use of a complex according to the invention in the formulation of a human insulin solution with a concentration of 100 IU/ml intended for implantable or transportable insulin pumps.

### EXAMPLE 1

### 100 IU/ml Fast-Acting Insulin Analog Solution

This solution is a commercial Novo solution sold under the name of Novolog. This product is a fast-acting insulin analog.

### EXAMPLE 2

### 100 IU/ml Human Insulin Solution

This solution is a commercial Novo solution sold under the name of Actrapid. This product is a human insulin.

### EXAMPLE 3

### Preparation of a 200 IU/ml Human Insulin Solution

125.6 mg of insulin (21.6 μmol) comprising 470 μg of Zn2+ are suspended in 8.74 ml of 40 mM acetic acid. The protein is subsequently dissolved by the addition of 1.35 ml of 0.1N HCl (pH 2.6).

The final concentration is subsequently adjusted to 200 IU/ml (1.2 mM) by addition of water.

The final pH of this solution is 2.6 for an acetic acid concentration of 20 mM.

This clear solution is filtered through a 0.22 μm filter.

### EXAMPLE 4

### Preparation of the Excipients

Preparation of the 200 mM Phosphate Buffer at pH 7

A solution A of monosodium phosphate is prepared as follows: 1.2 g of NaH2PO4 (10 mmol) are dissolved in 50 ml of water in a volumetric flask.

A solution B of disodium phosphate is prepared as follows: 1.42 g of Na2HPO4 (10 mmol) are dissolved in 50 ml of water in a volumetric flask.

The 200 mM phosphate buffer at pH 7 is obtained by mixing 3 ml of solution A with 7 ml of solution B.

Preparation of a 130 mM m-cresol Solution

The m-cresol solution is obtained by dissolving 0.281 g of m-cresol (2.6 mmol) in 20 ml of water in a volumetric flask.

Preparation of a 50 mM EDTA Solution

The EDTA solution is obtained by dissolving 0.372 g of EDTA (1 mmol) in 20 ml of water in a volumetric flask.

Preparation of a 0.8 mM Tween 20 Solution

The Tween 20 solution is obtained by dissolving 98 mg of Tween 20 (80 μmol) in 100 ml of water in a volumetric flask.

Preparation of a 1.5M glycerol Solution

The glycerol solution is obtained by dissolving 13.82 g of glycerol (150 mmol) in 100 ml of water in a volumetric flask.

Preparation of the Solutions of Amphiphilic Polymers

Two amphiphilic polymers are employed.

The polymer 1 is a sodium dextranmethylcarboxylate modified by the sodium salt of L-tryptophan obtained from a dextran with a weight-average molar mass of 10 kg/mol, i.e. a degree of polymerization of 39 (Pharmacosmos), according to the process described in patent application FRO7.02316. The molar fraction of sodium methylcarboxylate, modified or not modified by the tryptophan, i.e. i in the formula I, is 1.03. The molar fraction of sodium methylcarboxylate modified by the tryptophan, i.e. n in the formula I, is 0.36.

The solution of polymer 1 is obtained by dissolving 4.03 g of polymer 1 (water content=10%) in 15 ml of water in a 50 ml tube.

This solution is subsequently adjusted to pH 5.5 with a 0.1N HCl solution.

The solution of polymer 1 is decanted into a 25 ml volumetric flask and the concentration is adjusted to 145 mg/ml by making up to the graduation mark with water.

The polymer 2 is a sodium dextranmethylcarboxylate modified by the sodium salt of L-tryptophan obtained from a dextran with a weight-average molar mass of 40 kg/mol, i.e. a degree of polymerization of 154 (Pharmacosmos), according to the process described in patent application FR07.02316. The molar fraction of sodium methylcarboxylate, modified or not modified by the tryptophan, i.e. i in the formula I, is 1.03. The molar fraction of sodium methylcarboxylate modified by the tryptophan, i.e. n in the formula I, is 0.37.

The solution of polymer 2 is obtained by dissolving 4.03 g of polymer 2 (water content=10%) in 15 ml of water in a 50 ml tube.

This solution is subsequently adjusted to pH 5.5 with a 0.1N HCl solution.

The solution of polymer 2 is decanted into a 25 ml volumetric flask and the concentration is adjusted to 145 mg/ml by making up to the graduation mark with water.

### EXAMPLE 5

### Preparation of a 100 IU/ml Human Insulin Solution in the Presence of Polymer 1

For a final volume of 7 ml of formulation with a [polymer 1]/[insulin] ratio by weight of 1.0, the various reactants are mixed in the amounts specified in the table below and in the order which follows:

| | | |
|---|---|---|
| Insulin at 200 IU/ml | 3.5 | ml |
| EDTA at 50 mM | 28 | μl |
| Polymer 1 at 145 mg/ml | 174 | μl |
| Adjustment pH 7 with 1N NaOH | 95 | μl |
| Phosphate buffer, 200 mM, pH 7 | 350 | μl |
| Tween 20, 0.8 mM | 70 | μl |
| Glycerol, 1.5 M | 793 | μl |
| m-Cresol, 130 mM | 1.562 | ml |
| Water (Volume for dilution − volume of | 425 | μl |
| sodium hydroxide solution) | | |

The final pH is 7±0.3.

This clear solution is filtered through a 0.22 μm filter and is then placed at +4° C.

The examples from 6 to 8 were prepared according to the same procedure by varying the volumes of polymer solution, in order to achieve the polymer/insulin ratios by weight shown in the table, the type of polymer, the presence of EDTA, the type of antibacterial agents and/or the presence of Tween and glycerol. The final solutions have a pH of 7; they are isotonic, clear and filtered through a 0.22 μm filter.

| | | | Antibacterial | | |
|---|---|---|---|---|---|
| | | Polymer/insulin | agents | | EDTA |
| Example | Polymer | ratio by weight | (mM) | Excipients | (μM) |
| 5 | 1 | 1.0 | Cresol (29) | 8 μM | 200 |
| | | | | Tween, | |
| | | | | Glycerol | |
| 6 | 1 | 1.0 | Cresol (29) | 8 μM | 0 |
| | | | | Tween, | |
| | | | | Glycerol | |
| 7 | 1 | 2.1 | Phenol (29) | | 200 |
| 8 | 2 | 0.8 | Cresol (29) | 8 μM | 200 |
| | | | | Tween, | |
| | | | | Glycerol | |

### EXAMPLE 9

### Kinetics of Insulin Aggregation

The samples are placed on a rotor at room temperature. In the solution prepared in example 1, aggregates appear from the one hundredth hour. In the solution prepared in example 5, aggregates appear only from the three hundredth hour.

### EXAMPLE 10

### Injectability of the Solutions

All these solutions can be injected with the usual insulin injection systems. The solutions described in examples 1, 2 and 5 to 8 are injected just as easily with insulin syringes with 31 gauge needles. The solutions described in examples 1 to 2 and 5 to 8 are injected just as easily with the Novo insulin pen, sold under the name of Novopen, with 31 gauge needles.

### EXAMPLE 11

### Protocol for Measuring the Pharmacodynamics of the Insulin Solutions

6 domestic pigs weighing approximately 50 kg, catheterized beforehand at the jugular vein, are deprived of food 2 to 3 hours before the beginning of the experiment. In the hour preceding the injection of insulin, 3 blood samples are taken in order to determine the basal level of glucose and insulin.

Insulin is injected subcutaneously in the neck, under the ear of the animal, at a dose of 0.0625 IU/kg.

Blood samples are subsequently taken every 10 minutes over 3 hours and then every 30 minutes up to 5 hours. After each sample is taken, the catheter is rinsed with a dilute heparin solution.

A drop of blood is withdrawn in order to determine the blood glucose level using a glucometer.

The curves for glucose pharmacodynamics are subsequently plotted.

### EXAMPLE 12

### Pharmacodynamics Results for the Insulin Solutions

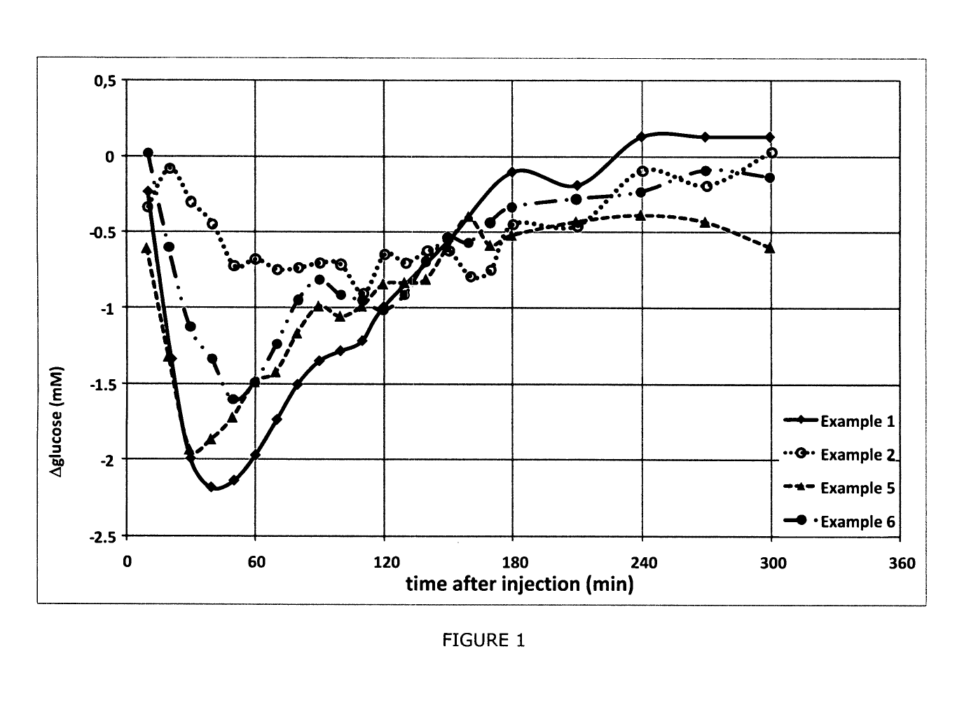

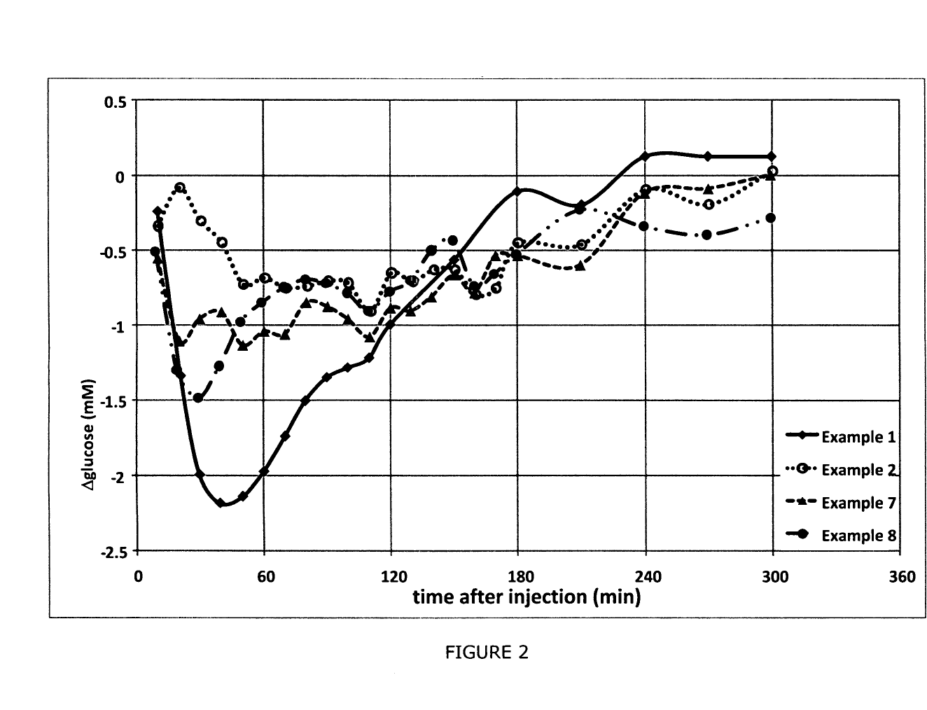

The results obtained, represented in the curves of FIGS. 1 and 2, show that all the formulations of the complex according to the invention (“example to 5 to 8” curves) make it possible to obtain an onset of action of less than 30 minutes and a glycemic nadir at less than 2 hours, which are systematically less than those of the human insulin formulations (“example 2” curve) and substantially comparable to those of the fast-acting insulin formulations (“example 1” curve).

1. A complex between human insulin and an amphiphilic polymer comprising carboxyl functional groups, said amphiphilic polymer being chosen from functionalized polysaccharides predominantly composed of glycoside monomers bonded via glycoside bonds of (1,6) type which are functionalized by at least one tryptophan derivative, which polysaccharides are chosen from the polysaccharides of formula I: 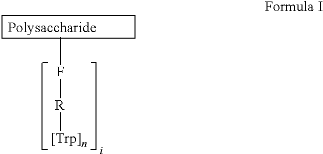
in which:
F resulting from the coupling between the connecting arm R and an —OH function of the polysaccharide and being either an ester, thionoester, amide, carbonate, carbamate, ether, thioether or amine function,
R being a chain comprising between 1 and 6 carbons, optionally branched and/or unsaturated, comprising one or more heteroatoms, and/or S, and having at least one carboxyl function,
Trp being a residue of an L or D tryptophan derivative, the product of the coupling between the amine of the tryptophan derivative and at least one acid carried by the R group and/or one acid carried by the polysaccharide comprising carboxyl functional groups,
n represents the molar fraction of the R groups substituted by Trp and is between 0.05 and 0.7,
i represents the molar fraction of the F-R-[Trp]ₙ groups carried per saccharide unit and is between 0 and 2,
and, when R is not substituted by Trp, then the acid or acids of the R group are carboxylates of a cation said polysaccharides being amphiphilic at neutral pH.

2. The complex as claimed in claim 1, the polysaccharide being a dextran.

3. The complex as claimed in claim 1, F being either an ester, a carbonate, a carbamate or an ether.

4. The complex as claimed in claim 1, the R group being chosen from the following groups: 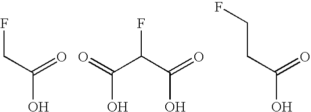
or their salts of alkali metal cations.

5. The complex as claimed in claim 1, the tryptophan derivative being chosen from the group consisting of tryptophan, tryptophanol, tryptophanamide, 2-indoleethylamine and their alkali metal cation salts.

6. The complex as claimed in claim 1, the tryptophan derivative being chosen from the tryptophan esters of formula II: 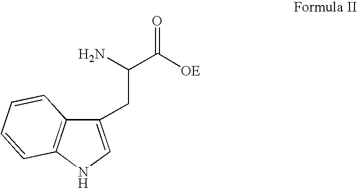
E being a linear or branched C₁ to C₄ alkyl group.

7. The complex as claimed in claim 1, the insulin being a recombinant human insulin.

8. The complex as claimed in claim 1, the polymer/insulin ratios by weight being between 0.1 and 5.

9. The complex as claimed in claim 1, the polymer/insulin ratios by weight being between 0.7 and 1.3.

10. A pharmaceutical composition comprising at least one complex as claimed in claim 1.

11. The composition as claimed in claim 10, which is in the form of an injectable solution.

12. The composition as claimed in claim 10, the concentration of the solutions being 600 μM, i.e. 100 IU/ml.

13. The use of a complex as claimed in claim 1 in the preparation of a human insulin formulation at a concentration of approximately 600 μM (100 IU/ml), the onset of action of which is less than 30 minutes.

14. A human insulin formulation comprising the complex as claimed in claim 1 at a concentration of approximately 600 μM (100 IU/ml), the glycemic nadir of which is at less than 120 minutes.

15. A 100 IU/ml insulin formulation intended for injection pumps comprising the complex as claimed in claim 1.
